(19) United States
(12) Patent Application Publication
(10) Pub. No.: US 2014/0039280 A1
(43) Pub. Date: Feb. 6, 2014

(54) Non-Invasive Diagnostic Device for Early Detection of Infection and Inflammation and Methods of Use

(71) Applicants: Barry W. Allen, Rougemont, NC (US); Claude A. Piantadosi, Durham, NC (US); Jeffrey Steven Meisner, Durham, NC (US); Stephen Lawrence Craig, Chapel Hill, NC (US)

(72) Inventors: Barry W. Allen, Rougemont, NC (US); Claude A. Piantadosi, Durham, NC (US); Jeffrey Steven Meisner, Durham, NC (US); Stephen Lawrence Craig, Chapel Hill, NC (US)

(73) Assignee: DUKE UNIVERSITY, Durham, NC (US)

(21) Appl. No.: 13/957,205

(22) Filed: Aug. 1, 2013

Related U.S. Application Data
(60) Provisional application No. 61/678,819, filed on Aug. 2, 2012.

(51) Int. Cl. A61B 5/00

(52) U.S. Cl. CPC A61B 5/412
USPC 600/309; 600/532

(57) ABSTRACT

The present disclosure provides, in part, a self-contained, point-of-care device for the early detection of infection, or other pathologic conditions that can lead to system inflammation, by the detection of the endogenous gaseous mediator nitric oxide (NO.) and methods of use.

### FEDERAL FUNDING LEGEND

This invention was produced in part using funds from the Federal Government under Office of Naval Research Grant No.: N00014-01-1-0240 entitled “Nitric Oxide and CNS O₂ Toxicity: Biochemical Modeling” and subsequent renewals thereof. Accordingly, the Federal Government has certain rights to this invention.

### BACKGROUND

Sepsis is a potentially deadly medical condition that is characterized by a whole-body inflammatory state (also known as systemic inflammatory response syndrome or SIRS) that is triggered by an infection. Sepsis often arises because of an undetected and/or untreated infection. Sepsis can also complicate certain injuries and diseases. Sepsis is the 10^{th} leading cause of death in the United States (13), where more than 750,000 cases occur each year, resulting in the loss of more than 200,000 lives and a cost of $16.7 billion (2). World-wide, the toll is much greater.

Complications from inflammation also play a role in other conditions, such as transplantation. For example, acute rejection of an allograft—an implanted tissue or organ from a genetically non-identical donor—involves an inflammatory response that shares some biochemical features with the response to infection (5, 15).

The early detection of infection, to permit anti-infective treatment before a systemic response has occurred, is critical to reducing deaths resulting from sepsis; and early detection of inflammation, with or without infection, is useful in those cases involving allograft rejection, so that timely immunosuppressive therapy can be provided (11).

Hence, an inexpensive, non-invasive device and method is needed to alert medical personnel to the onset of infection or significant inflammation in vulnerable patients, in order to permit swift prophylactic treatment.

### SUMMARY OF THE INVENTION

The present disclosure provides, in part, a self-contained, point-of-care device for the early detection of infection, or other pathologic conditions that can lead to systemic inflammation, by the detection of the endogenous gaseous mediator nitric oxide (NO.).

One aspect of the present disclosure provides a noninvasive diagnostic device placed in contact with the skin or tissue of a subject comprising a detector capable of detecting the production of nitric oxide (NO.) by inflammatory cells to provide early warning of the onset of infection.

Another aspect of the present disclosure provides a non-invasive diagnostic device placed in contact with the skin or tissue of a subject comprising a detector capable of detecting the production of nitric oxide (NO.) by inflammatory cells to provide early warning of the onset of acute allograft rejection.

Yet another aspect of the present disclosure provides a non-invasive device placed in contact with the exhaled breath of a subject comprising a detector capable of detecting the production of nitric oxide (NO.) by inflammatory cells to provide early warning of the onset of pneumonia or other respiratory infection or inflammation.

In one embodiment, the detector further comprises an adherent patch or strip, the patch or strip being placed in contact with the skin at or near a site selected from the group consisting of surgical sites, sites of invasive monitoring, entry or exit sites of venous or arterial catheters or surgical drainage tubes, or any other superficial site likely to be at or near the seat of infection or inflammation.

In another embodiment, the detector further comprises an adherent patch or strip placed in contact with the skin in any well-vascularized region, the device being capable of detecting nitric oxide released into the bloodstream from a remote site of infection or inflammation.

In another embodiment, the device further comprises an adherent patch or strip placed in contact with the skin at or near the site of an allograft, the device being capable of detecting nitric oxide produced in the early stages of acute allograft rejection.

In some embodiments, the detector is embedded in the adherent patch or strip. In other embodiments, the detector is in a form that can be incorporated in a ventilator circuit in contact with the exhaled breath.

In another embodiment, the detector is in a form that can be incorporated in an arterial or venous catheter, in a urinary catheter, or in a surgical drainage tube.

In another embodiment, the device is selectively sensitive to nitric oxide (NO.) or derivatives thereof. In certain embodiments, the NO. derivatives are selected from the group consisting of oxidation products or metabolites of NO. (collectively termed NOₓ and including but not limited to NO⁺, NO⁻, NO₂, NO₂⁻, NO₃⁻, OONO⁻ or nitrotyrosine), or one or more of its adducts or compounds, including but not limited to nitrosothiols (R—SNO) or combinations thereof.

In another embodiment, the device further comprises a visual display of the detection of NO. or its derivatives, generated either by a chromogenic chemical reaction (using for example a diacetylene monomer or a conjugated polydiacetylene, any of which may be functionalized with other chemical compounds or complexed with a metal) or the chemical activation of the mesogenic component of a liquid crystal display.

In another embodiment, the device further comprises a chemical amplification means by which the sensitivity to nitric oxide-derived chemical species is enhanced through chemical amplification. In some embodiments, the chemical amplification comprises the use of encapsulated catalytic reagents that are released in the presence of NO. or derivatives thereof.

In another embodiment, the device further comprises a fluid medium having a high solubility for NO. and derivatives thereof, the fluid medium creating a favorable gradient for diffusion of NO. or derivatives thereof to the detector. In some embodiments, the fluid medium comprises a fluorocarbon or a fluorocarbon emulsion. In certain embodiments, the fluorocarbon emulsion comprises Oxycite®.

In another embodiment, the surfaces of the device that are not in contact with the skin comprise a barrier membrane to prevent the exchange of gases with the ambient atmosphere.

In yet another embodiment, the visual display, once activated, persists for sufficient time to allow observation by medical personnel, at least for two hours.

Another aspect of the present disclosure provides a method of detecting the production of nitric oxide (NO.) by inflammatory cells to provide early warning of the onset of infection in a subject comprising (a) placing the detection device according to the present disclosure against the skin or tissue of the subject; (b) detecting the presence or absence of nitric oxide (NO.); and (c) administering a therapeutically effective amount of an anti-infective agent(s) to the subject if nitric oxide is detected.

Another aspect of the present disclosure provides a method of detecting the production of nitric oxide (NO.) by inflammatory cells to provide early warning of the onset of acute allograft rejection in a subject comprising (a) placing the detection device according to the present disclosure against the skin or tissue of the subject; (b) detecting the presence or absence of nitric oxide (NO.); and (c) administering a therapeutically effective amount of an anti-inflammatory agent(s) to the subject if nitric oxide (NO.) is detected.

Another aspect of the present disclosure provides a method of detecting the production of nitric oxide (NO.) by inflammatory cells in the breath of a subject comprising (a) placing the detection device according to the present disclosure such that the device is in contact with the breath of the subject; (b) detecting the presence or absence of nitric oxide (NO.); and (c) administering a therapeutically effective amount of an anti-infective agent(s) to the subject if nitric oxide (NO.) is detected.

### BRIEF DESCRIPTION OF THE DRAWINGS

The foregoing aspects and other features of the disclosure are explained in the following description, taken in connection with the accompanying drawings, wherein:

FIG. 1 is a graph showing lipopolysaccharide (LPS, 1 mg/kg) injected into the peritoneal cavity of an anesthetized rat (arrow), elicits a strong signal at an NO. selective electrode in the vena cava.

FIG. 2 is a graph showing the detection of pathological levels of NO. in vivo. An intravascular nitric oxide sensor was inserted in a femoral vein in an anesthetized baboon (Papio cynocephlus). Heat-killed bacteria (E. coli) were infused into a brachial vein (arrow). Approximately 20 min later, the output of a ruthenium electrode demonstrated a strong signal consistent with nitric oxide production by the animal in response to the experimental bacteremia.

FIG. 3 illustrates the structure of 10,12-pentacosadiynioc acid monomer, full structure (left) and simplified structure (right).

FIG. 4 is a UV-Vis spectra of the colorless monomer film (m), the photopolymerized film (blue), and thermally-treated red film (red).

FIG. 5 illustrates the chemical structure of the diacetylene monomer film and its subsequent polymerization to a polydiacetylene (PDA) film initiated by exposure to NO. gas (NO_{(g)}).

FIG. 6 is a schematic diagram of the monomer film design showing the color change (colorless to blue) induced by the polymerization of monomer films with NO. gas (NO_{(g)}) and/or its oxidation products.

FIG. 7 is a photographic scan of monomer film (left) and polymer film (right), demonstrating color change resulting from exposure of the monomer to NO..

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

Unless otherwise defined, all technical terms used herein have the same meaning as commonly understood by one of ordinary skill in the art to which this disclosure belongs.

The present disclosure provides, in part, a self-contained, point-of-care device for the early detection of infection, or other pathologic conditions that can lead to systemic inflammation, by the detection of the endogenous gaseous mediator nitric oxide (NO.). Nitric oxide (NO.) is a gaseous free radical produced in the body that serves at least two classes of functions. Under normal physiological conditions, it is produced at low concentrations and serves as a signaling molecule that regulates many functions, including the contractile state of the vasculature. However, in the event of infection or other conditions that trigger acute inflammation, NO. is transiently released into the bloodstream at much higher concentrations; and since NO. is preferentially soluble in lipids, it diffuses through tissue and may be detected at some distance from the site of infection or injury, for example at the skin. In some cases it, or its oxidation products, can be detected in exhaled breath.

A. Nitric Oxide in the Skin.

Physical and pathophysiological processes can account for the appearance of NO. in the skin in response to infection. First, nitric oxide is 3-4 times more soluble in lipids than in aqueous phases (6) and therefore partitions selectively into biological membranes from remote sites of production in aqueous extracellular fluids, including blood plasma. Second, the enzyme nitric oxide synthase 2 (NOS2), also called inducible NOS or iNOS, which produces nitric oxide at high concentrations after infection or tissue damage, is found in macrophages, specialized cells mobilized as part of the immune response; and macrophages are found in many tissues, including the skin (12). Third, NOS2 is not limited to macrophages, but has been detected in virtually all cell types, including other kinds of skin cells (4). So it is possible that NO can be produced directly in the skin by NOS2 in response to cytokines, chemical signals whose concentrations are upregulated by several orders of magnitude in response to infection or trauma. Third, fibroblasts from human skin produce NO. linked to another enzyme responsible for NO. synthesis, calcium-dependent endothelial nitric oxide synthase, also called eNOS or NOS3 (3). Release of NO. by NOS3 is induced by elevated calcium levels and terminates within minutes as those levels return to baseline. Other cell types known to produce NO. rapidly in response to certain chemical signals include many endothelial cell types, some neurons, peripheral blood neutrophils, and mast cells (7); and there are mast cells in the skin. Further, the addition of LPS, a component of some bacterial cell walls and a marker for infection, induces a rapid elevation of calcium levels which reaches maximal levels within minutes and slowly returns to baseline values (10), consistent with the time course of the transient NO. signal (see Examples).

B. Distinguishing Infection from Trauma.

Nitric oxide release has been detected in response to both infection and to aseptic tissue injury. In a device intended to provide early diagnosis of infection, it would be important to be able to distinguish between these two conditions. Studies in which oxidation products of NO. have been assessed, show that NO. production was significantly higher in sepsis than in trauma without infection (8, 14). Thus, careful calibration of the chromogenic response to NO. should allow infection to be discriminated from the differential diagnosis of aseptic injury.

Accordingly, the present disclosure relates to non-invasive detection of concentrations of nitric oxide (NO.) or related chemical species produced by inflammatory cells in a subject due to the onset of infection or inflammation. Such detection is early enough in the inflammatory process to provide early warning to a medical care provider, thereby allowing for therapeutic treatment of the infection and/or inflammatory response to begin.

As used herein, the term “inflammatory cells” refers to any of those cells involved in defending the body against both infectious disease and foreign materials. Such cells include, but are not limited to, neutrophils, eosinophils, basophils, B and T lymphocytes, monocytes, including macrophages, and the like.

As used herein, the term “subject” is intended to include human and non-human animals. Exemplary human subjects include a human patient suffering from an infection with a pathogen (e.g., infectious disease), inflammation due to said infectious disease, undergoing an allogeneic transplant, and the like. The term “non-human animals” includes all vertebrates, e.g., non-mammals (such as chickens, amphibians, reptiles) and mammals, such as non-human primates, domesticated and/or agriculturally useful animals (such as sheep, dogs, cats, cows, pigs, etc.), and rodents (such as mice, rats, hamsters, guinea pigs, etc.).

As used herein, the term “infectious disease” refers to any disease caused by an infectious agent (e.g., virus, bacteria, parasite, yeast, fungi and the like) that has, as one of its symptoms or side-effects, inflammation. The term “pathogen” or “infectious agent” refers to any microorganism, such as a virus, bacteria, parasite, yeast, fungi and the like, that causes disease in its host.

In some embodiments, the detector is an adherent patch or strip, where the patch or strip is placed in contact with the skin or tissue at or near a desired site. In other embodiments, the device is embedded in the patch or strip. In some embodiments, the patch or strip is disposable. In other embodiments, the device is reusable and can be removed from the disposable patch or strip. Suitable sites can be determined by one skilled in the art and will depend on the condition of the subject being treated. Examples of a suitable site may include, but are not limited to, surgical sites, sites of invasive monitoring, entry or exit sites of venous or arterial catheters, or any other superficial site likely to be at or near the seat of infection or inflammation, any well-vascularized region, and/or at or near the site of an allograft.

In some embodiments, the detector is embedded in the adherent patch or strip. In other embodiments, the detector is in a form that can be incorporated in a ventilator circuit in contact with the exhaled breath, or incorporated in venous, arterial or urinary catheters or surgical drainage tubes.

Broadly, the device is selectively sensitive to nitric oxide (NO.) or derivatives thereof. Suitable NO derivatives include, but are not limited to, metabolites of NO. (collectively termed NOₓ and including but not limited to NO⁺, NO⁻, NO₂, NO₂⁻, NO₃⁻, OONO⁻ or nitrotyrosine), or one or more of its adducts or compounds, including but not limited to nitrosothiols (R—SNO) or combinations thereof.

In some embodiments, the device further comprises a visual display of the detection of NO. or its derivatives generated either by a chromogenic chemical reaction or a colorimetric chemical reaction (using for example a diacetylene monomer or a conjugated polydiacetylene, any of which may be functionalized with other chemical compounds or complexed with a metal) or the chemical activation of the mesogenic component of a liquid crystal display. In particular examples, NO. is detected by a device that comprises a monomer that is polymerized by NO. and results in a color change of the detector.

Even though nitric oxide is released at relatively high concentrations in infection or inflammation, it is a fugitive and reactive molecule, and its concentrations may be attenuated in passing through the skin or tissue from a site of infection or inflammation. To address this, other embodiments of the present disclosure provide a device further comprising a chemical amplification means by which the sensitivity to nitric oxide-derived chemical species is enhanced through chemical amplification. In some embodiments, the chemical amplification comprises the use of encapsulated catalytic reagents that are released in the presence of NO. or derivatives thereof.

In other embodiments, the device further comprises a fluid medium (e.g., a solvent) having a high solubility for NO. and derivatives thereof. In such embodiments, the fluid medium creates a favorable gradient for diffusion of NO. or derivatives thereof from the skin or tissue to the detector. Examples of suitable solvents include, but are not limited to, a fluorocarbon compound or a fluorocarbon emulsion, such as Oxycite®.

In other embodiments, the surface(s) of the device that are not in contact with the skin, tissue or exhaled breath comprise(s) a barrier membrane to prevent the exchange of gases with the ambient atmosphere, thereby limiting the dilution or loss of NO-derived chemical species, their destruction by chemical reaction with components of the atmosphere, and/or the intrusion of NO. or its derivatives from exogenous sources. Suitable examples of such barriers include PET (poly[ethylene terephthalate]) coated with layers of cationic polyethylenimine (PEI) and anionic montmorillonite clay (MMT) and poly[acrylic acid] (PAA), as have been developed to meet the needs of the food-packaging industry (9).

In yet another embodiment, the visual display, once activated, persists for sufficient time to allow observation by medical personnel. A suitable amount of time may include 10 min, 20 min, 30 min, 45 min, 1 hour, 2 hours, 4 hours, 6 hours, 12 hours and 24 hours, or longer. In certain embodiments, the visual display lasts for at least for two hours.

For a simple, inexpensive and disposable device, other embodiments provide that infection or inflammation is detected with a chemical system that generates a color change in response to the presence of NO., and therefore would not require connection to ancillary equipment, such as the electronic apparatus needed to support an electrochemical sensor.

Another aspect of the present disclosure provides a method of detecting the production of nitric oxide (NO.) by inflammatory cells to provide early warning of the onset of infection in a subject comprising (a) placing the detection device according to the present disclosure against the skin or tissue of the subject; (b) detecting the presence or absence of nitric oxide (NO.); and (c) administering a therapeutically effective amount of an anti-infective agent(s) to the subject if nitric oxide is detected.

Another aspect of the present disclosure provides a method of detecting the production of nitric oxide (NO.) by inflammatory cells to provide early warning of the onset of acute allograft rejection in a subject comprising (a) placing the detection device according to the present disclosure against the skin of the subject; (b) detecting the presence or absence of nitric oxide (NO.); and (c) administering a therapeutically effective amount of an anti-inflammatory agent(s) to the subject if nitric oxide (NO.) is detected.

Another aspect of the present disclosure provides a method of detecting the production of nitric oxide (NO.) by inflammatory cells in the exhaled breath of a subject comprising (a) placing the detection device according to the present disclosure such that the device is in contact with the exhaled breath of the subject; (b) detecting the presence or absence of nitric oxide (NO.); and (c) administering a therapeutically effective amount of an anti-infective agent(s) to the subject if nitric oxide (NO.) is detected.

As used herein, the term “therapeutically effective” refers to a dosage of a compound (e.g., an anti-infective and/or anti-inflammatory agent) that is effective for eliciting a desired effect. This term as used herein may also refer to an amount effective at bringing about a desired in vivo effect in an animal, mammal, or human, such as reducing hemorrhaging, inflammation, fever and the like. A therapeutically effective amount may be administered in one or more administrations (e.g., the agent may be given as a preventative treatment or therapeutically at any stage of disease progression, before or after inflammatory symptoms, and the like), in any means of application or dosages and is not intended to be limited to a particular formulation, combination or administration route. It is within the scope of the present disclosure that the agents may be administered at various times during the course of infection of the subject. The times of administration and dosages used will depend on several factors, such as the goal of treatment (e.g., treating v. preventing), condition of the subject, etc. and can be readily determined by one skilled in the art. For example, in one embodiment the agent is administered at the onset of infection (i.e., when NO. is detected). In other embodiments, the agent is administered prior to the onset of infection (i.e., before NO. is detected). The term “administration” or “administering,” as used herein, refers to providing, contacting, and/or delivery of an agent by any appropriate route to achieve the desired effect. These compounds may be administered to a subject in numerous ways including, but not limited to, orally, ocularly, nasally, intravenously, intramuscularly, intraperitoneally, intrathecally, topically, as aerosols, suppository, etc. and may be used in combination.

The devices described herein have the potential to save thousands of lives in civilian as well as in military medical practice. Furthermore, the early treatment of sepsis and other severe infections, such as ventilator associated pneumonia (VAP) or other hospital-acquired pneumonias, has been shown to significantly reduce the duration and cost of treatment.

The inherent simplicity of the disclosed devices, and methods of using the devices, could lead to worldwide adoption, not only in clinical settings but in field conditions as well. The detection of infection or severe inflammation at the earliest possible stages, and intervention with appropriate treatment, greatly reduces mortality and healthcare costs.

The following examples are offered by way of illustration and not by way of limitation.

### EXAMPLES

### Example 1

Early Detection of Host Response to Experimental Sepsis.

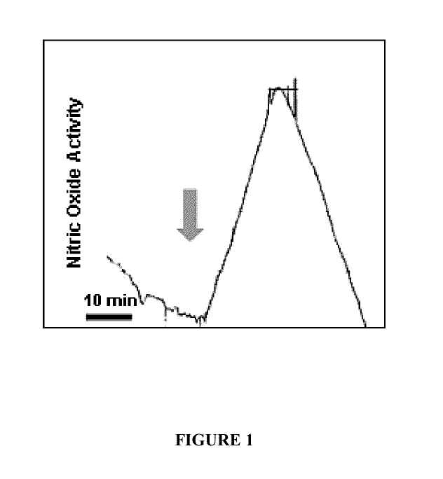

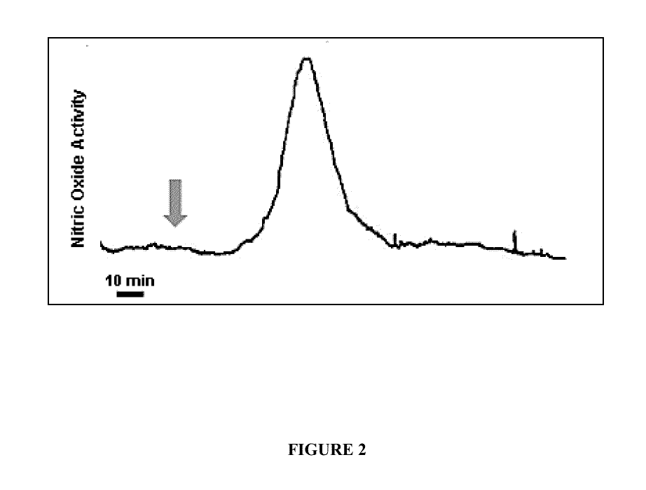

Intra-vascular response. This experiment demonstrated that a host response (a natural defensive reaction of the body to infection) to early experimental sepsis was detected minutes after the introduction of bacterial components or whole bacteria in rat (FIG. 1) and baboon (FIG. 2). Specifically, nitric oxide was measured in the vascular compartment following introduction of bacterial components using the electrochemical method disclosed in U.S. Pat. Nos. 5,980,705, 6,287,452 and 6,280,604, all herein incorporated by reference in their entirety. Detection of NO. was far earlier, by many hours, than conventional culture methods for detecting the onset of infection. That these responses were authentic nitric oxide signals was corroborated by the fact that they were abolished when an inhibitor of nitric oxide synthesis, N^{G}-nitro-L-arginine methyl ester (L-NAME), was administered 30 minutes prior to the introduction of the infectious agent. In addition, in vitro studies showed that this sensor was highly specific for nitric oxide and relatively insensitive to substances that would likely be present in biological systems and that could produce a signal at the electrode and thus be confounded with nitric oxide (1). Thus, this experiment illustrates that the nitric oxide signal by activated inflammatory cells is a characteristic of the host response to the onset of infection and/or inflammation including the early stages of all microbial infections—whether the agent is bacterial, viral or fungal.

Transdermal Detection. A nitric oxide sensor according to one embodiment of the present disclosure may be used to detect a nitric oxide host response on the surface of the skin (i.e., transdermal detection). Thus, a device according to one embodiment of the present disclosure, when placed on the skin and visible to medical personnel, incorporates components for diagnostic detection and display in a single, integrated system. This point-of-care device provides a non-invasive, real-time indication of the NO. dependent host response.

### Example 2

Preparation and Testing of Diacetylene as a Means for Detecting Nitric Oxide.

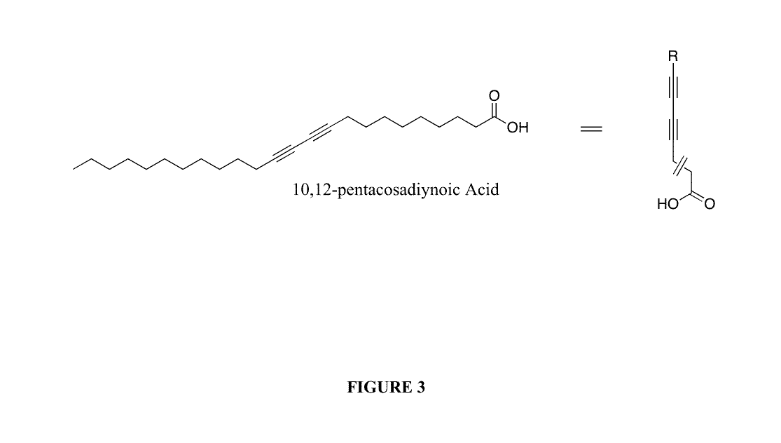

To prepare a diacetylene monomer, 10,12-pentacosadiynoic acid (purchased from Alfa Aesar, Ward Hill, Mass., USA; product number: L09313; CAS: 66990-32-7)) (FIG. 3) was dissolved in chloroform (CHCl₃; VWR International, Radnor, Pa., USA) and purified by filtration through a 0.2 micrometer syringe filter before use.

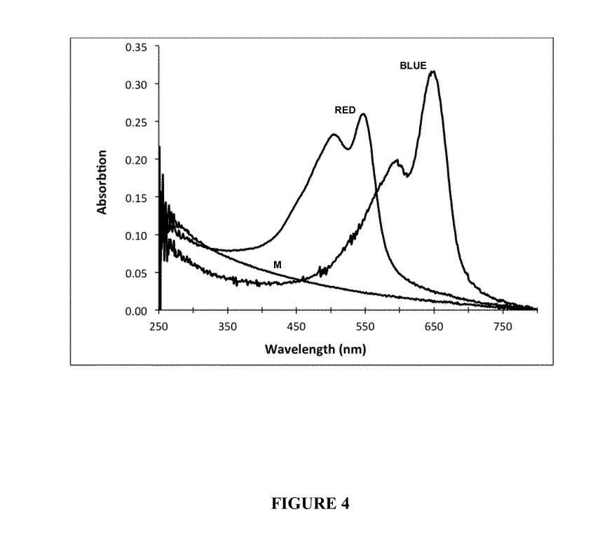

The 10,12-pentacosadiynoic acid in chloroform (0.190 g in 20 mL) was dropcast onto glass microscope slides. Evaporation of the chloroform solvent resulted in a colorless film. Chemical identity of the film was confirmed by ultraviolet-visible spectroscopy (UV-Vis) (FIG. 4).

To check the quality of the monomer films, photopolymerization was tested with UV-light as described in (16). Specifically, the monomer films were irradiated with 254 nm light for 5 min. The films turned from colorless to blue, and a characteristic spectrum was obtained (FIG. 4).

As a further test of quality, the thermal isomerization of the polymer was tested by quickly heating the PDA films (blue) under normal atmospheric conditions. The films slowly turn red in color (FIG. 4).

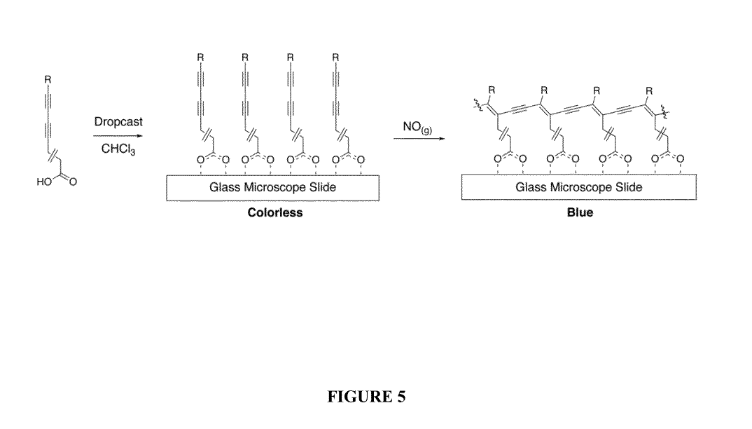

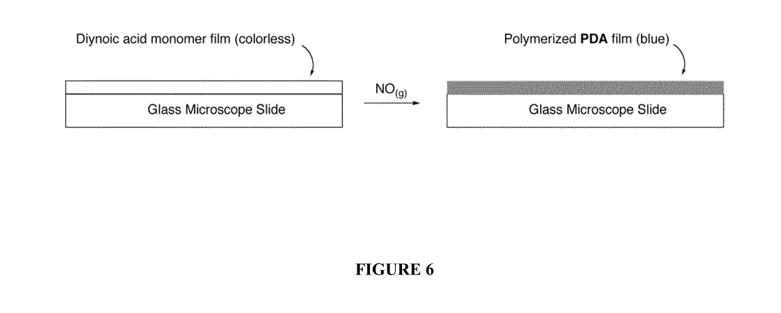

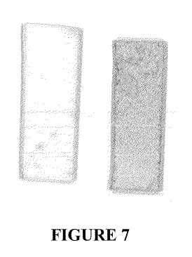

To determine if the monomer films (colorless) could be polymerized by exposure to nitric oxide, samples of dropcast monomer films were exposed to 10% NO. in nitrogen (N₂) for 10 min in a glass reaction chamber. The characteristic colorless-to-blue transition was observed (FIGS. 5-7), confirming that a diacetylene film can be used to detect the presence of NO. or its oxidation product.

### REFERENCES

1. Allen B W, Liu J, and Piantadosi C A. Electrochemical detection of nitric oxide in biological fluids. In: Methods in enzymology, nitric oxide, part e (Volume 396 ed.): Academic Press, 2005, p. 68-77.

2. Angus D, Linde-Zwirble W, Lidicker J, Clermont G, Carcillo J, and Pinsky M. Epidemiology of severe sepsis in the united states: Analysis of incidence, outcome, and associated costs of care. Critical Care Medicine 29: 1303-1310, 2001.

3. Frank S, Kampfer H, Wetzler C, and Pfeilschifter J. Nitric oxide drives skin repair: Novel functions of an established mediator. Kidney Int 61: 882-888, 2002.

4. Galea E and Feinstein D L. Regulation of the expression of the inflammatory nitric oxide synthase (nos2) by cyclic amp. The FASEB Journal 13: 2125-2137, 1999.

5. Langrehr J M, Murase N, Markus P M, Cai X, Neuhaus P, Schraut W, Simmons R L, and Hoffman R A. Nitric oxide production in host-versus-graft and graft-versus-host reactions in the rat. J Clin Invest 90: 679-683, 1992.

6. Moller M, Botti H, Batthyany C, Rubbo H, Radi R, and Denicola A. Direct measurement of nitric oxide and oxygen partitioning into liposomes and low density lipoprotein. Journal of Biological Chemistry 280: 8850-8854, 2005.

7. Nathan C. Nitric oxide as a secretory product of mammalian cells. The FASEB Journal 6: 3051-3064, 1992.

8. Ochoa J B, Udekwu A O, Billiar T R, Curran R D, Cerra F B, Simmons R L, and Peitzman A B. Nitrogen oxide levels in patients after trauma and during sepsis. Ann Surg 214: 621-626, 1991

9. Priolo M A, Gamboa D, Holder K M, and Grunlan J C. Super gas barrier of transparent polymer-clay multilayer ultrathin films. Nano Letters 10: 4970-4974, 2010.

10. Sun J, Zhou Z Q, Lv R, Li W Y, and Xu J G. Ketamine inhibits 1 ps-induced calcium elevation and nf-kappa b activation in monocytes. Inflammation Research 53: 304-308, 2004.

11. United Network for Organ Sharing. Costs. In: Transplant Living™, Your prescription for transplant information: United Network for Organ Sharing, 2011.

12. von Stebut E, Belkaid Y, Nguyen B, Wilson M, Sacks D L, and Udey M C. Skin-derived macrophages from leishmania major-susceptible mice exhibit interleukin-12-and interferon-[ggr]-independent nitric oxide production and parasite killing after treatment with immunostimulatory DNA. 119: 621-628, 2002.

13. Wisplinghoff H, Bischoff T, Tallent S M, Seifert H, Wenzel R P, and Edmond M B. Nosocomial bloodstream infections in us hospitals: Analysis of 24,179 cases from a prospective nationwide surveillance study.(major article). Clinical Infectious Diseases 39: 309(309), 2004.

14. Wong H R, Carcillo J A, Burckart G, and Kaplan S S. Nitric oxide production in critically ill patients. Archives of Disease in Childhood 74: 482-489, 1996.

15. Yang X, Chowdhury N, Cai B, Brett J, Marboe C, Sciacca R R, Michler R E, and Cannon P J. Induction of myocardial nitric oxide synthase by cardiac allograft rejection. The Journal of Clinical Investigation 94: 714-721, 1994.

16. Sun, X.; Chen, T.; Huang, S., Li, L; Peng, H. “Chromatic polydiacetylene with novel sensitivity”. Chem. Soc. Rev., 39, 4244-4257, 2010.

Any patents or publications mentioned in this specification are indicative of the levels of those skilled in the art to which the invention pertains. These patents and publications are herein incorporated by reference to the same extent as if each individual publication was specifically and individually indicated to be incorporated by reference. In case of conflict, the present specification, including definitions, will control.

One skilled in the art will readily appreciate that the present invention is well adapted to carry out the objects and obtain the ends and advantages mentioned, as well as those inherent therein. The present disclosure described herein are presently representative of preferred embodiments, are exemplary, and are not intended as limitations on the scope of the invention. Changes therein and other uses will occur to those skilled in the art which are encompassed within the spirit of the invention as defined by the scope of the claims.

We claim:

1. A noninvasive diagnostic device placed in contact with the skin or tissue of a subject, comprising a detector capable of detecting the production of nitric oxide (NO.) by inflammatory cells to provide early warning of the onset of infection.

2. A noninvasive diagnostic device placed in contact with the skin or tissue of a subject, comprising a detector capable of detecting the production of nitric oxide (NO.) by inflammatory cells to provide early warning of the onset of acute allograft rejection.

3. A noninvasive device placed in contact with the exhaled breath of a subject, comprising a detector capable of detecting the production of nitric oxide (NO.) by inflammatory cells to provide early warning of the onset of pneumonia or other respiratory infection or inflammation.

4. The noninvasive diagnostic device as in claim 1, in which the detector further comprises an adherent patch or strip, the patch or strip being placed in contact with the skin or tissue at or near site selected from the group consisting of surgical sites, sites of invasive monitoring, entry or exit sites of venous or arterial catheters or surgical drainage tubes, or any other superficial site likely to be at or near the seat of infection or inflammation.

5. The noninvasive diagnostic device as in claim 1, in which the detector further comprises an adherent patch or strip placed in contact with the skin or tissue in any well-vascularized region, the device being capable of detecting nitric oxide released into the bloodstream from a remote site of infection or inflammation.

6. The noninvasive diagnostic device of claim 2 further comprising an adherent patch or strip placed in contact with the skin or tissue at or near the site of an allograft, the device being capable of detecting nitric oxide produced in the early stages of acute allograft rejection.

7. The device as in claim 4, in which the detector is embedded in the adherent patch or strip.

8. The noninvasive diagnostic device of claim 3, the detector being in a form that can be incorporated in a ventilator circuit in contact with the exhaled breath.

9. The noninvasive diagnostic device as in claim 1, in which the device is selectively sensitive to nitric oxide (NO.) or derivatives thereof.

10. The device according to claim 9, wherein NO. derivatives are selected from the group consisting of oxidation products or metabolites of NO. or one or more of its adducts or compounds.

11. The device according to claim 10 wherein the oxidation products or metabolites of NO. are selected from the group consisting of NO⁺, NO⁻, NO₂, NO₂⁻, NO₃⁻, OONO⁻ or nitrotyrosine.

12. The device according to claim 10 wherein the adducts or compounds are selected from the group consisting of nitrosothiols (R—SNO) or combinations thereof.

13. The noninvasive diagnostic device of claim 9, further comprising a visual display of the detection of NO. or its derivatives, generated by a chromogenic chemical reaction or the chemical activation of a mesogenic component of a liquid crystal display.

14. The device according to claim 13 wherein the chromogenic chemical reaction comprises the use of a diacetylene monomer or a conjugated polydiacetylene.

15. The noninvasive diagnostic device as in claim 9, in which the device further comprises a chemical amplification means by which the sensitivity to nitric oxide-derived chemical species is enhanced through chemical amplification.

16. The device according to claim 15, wherein the chemical amplification comprises the use of encapsulated catalytic reagents that are released in the presence of NO. or derivatives thereof.

17. The noninvasive diagnostic device as in claim 9, in which the device further comprises a fluid medium having a high solubility for NO. and derivatives thereof, the fluid medium creating a favorable gradient for diffusion of NO. or derivatives thereof to the detector.

18. The device according to claim 17, wherein the fluid medium comprises a fluorocarbon or fluorocarbon emulsion.

19. The device according to claim 18, wherein the fluorocarbon emulsion comprises Oxycite®.

20. The noninvasive diagnostic device as in claim 9 in which the surfaces of the device that are not in contact with the skin comprise a barrier membrane to prevent the exchange of gases with the ambient atmosphere.

21. The noninvasive diagnostic device as in claim 9, wherein the visual display, once activated, persists for sufficient time to allow observation by medical personnel, at least for two hours.

22. A method of detecting the production of nitric oxide (NO.) by inflammatory cells to provide early warning of the onset of infection in a subject comprising:
a. placing the detection device according to claim 1 against the skin or tissue of the subject;
b. detecting the presence or absence of nitric oxide (NO.); and
c. administering a therapeutically effective amount of an anti-infective agent(s) to the subject if nitric oxide is detected.

23. A method of detecting the production of nitric oxide (NO.) by inflammatory cells to provide early warning of the onset of acute allograft rejection in a subject comprising:
a. placing the detection device according to claim 2 against the skin or tissue of the subject;
b. detecting the presence or absence of nitric oxide (NO.); and
c. administering a therapeutically effective amount of an anti-inflammatory agent(s) to the subject if nitric oxide (NO.) is detected.

24. A method of detecting the production of nitric oxide (NO.) by inflammatory cells in the breath of a subject comprising:
a. placing the detection device according to claim 3 such that the device is in contact with the exhaled breath of the subject;
b. detecting the presence or absence of nitric oxide (NO.); and
c. administering a therapeutically effective amount of an anti-infective agent(s) to the subject if nitric oxide (NO.) is detected.
